(19) United States
(12) Patent Application Publication
(10) Pub. No.: US 2025/0221816 A1
(43) Pub. Date: Jul. 10, 2025

(54) Ophthalmic Lens with Optimized Gusset Profile

(71) Applicant: Alcon Inc., Fribourg (CH)

(72) Inventor: Raza Shah, San Francisco, CA (US)

(21) Appl. No.: 18/978,444

(22) Filed: Dec. 12, 2024

Related U.S. Application Data
(60) Provisional application No. 63/618,061, filed on Jan. 5, 2024.

(51) Int. Cl. A61F 2/16

(52) U.S. Cl. CPC A61F 2/16; A61F 2002/1686; A61F 2230/0013

(57) ABSTRACT

A lens for implanting into an eye, which may comprise an optic having an optical axis, an optic edge defined by a first arc having a first center that is coincident with the optical axis, and a haptic junction defined by a second arc having a second center. The optical axis may be located between the second center and the second arc. A gusset may be coupled to the haptic junction, and a haptic may be coupled to the gusset. The gusset may have a thickness that increases between the haptic junction and the haptic. The optic edge may intersect the haptic junction. Additionally, or alternatively, the thickness of the gusset may increase monotonically with distance from the optical axis. In some embodiments, the thickness of the gusset may increase linearly with distance from the optical axis.

### PRIORITY CLAIM

This application claims the benefit of priority of U.S. Provisional Patent Application Ser. No. 63/618,061 titled “OPHTHALMIC LENS WITH OPTIMIZED GUSSET PROFILE”, filed on Jan. 5, 2024, whose inventor(s) is/are Raza Shah, which is hereby incorporated by reference in its entirety as though fully and completely set forth herein.

### TECHNICAL FIELD

The invention set forth in the appended claims relates generally to ophthalmic lenses, including, without limitation, intraocular lenses.

### BACKGROUND

The human eye can suffer a variety of maladies causing mild deterioration to complete loss of vision. While contact lenses and eyeglasses can compensate for some ailments, ophthalmic surgery may be required for others. In some instances, implants may be beneficial or desirable. For example, an intraocular lens may replace a clouded natural lens within an eye to improve vision.

While the benefits of intraocular lenses and other implants are known, improvements to lenses, delivery systems, components, and processes continue to improve outcomes and benefit patients.

### BRIEF SUMMARY

New and useful systems, apparatuses, and methods for eye surgery are set forth in the appended claims. Illustrative embodiments are also provided to enable a person skilled in the art to make and use the claimed subject matter.

For example, some embodiments comprise a lens having a circular optic edge with a gusset region that does not follow the shape of the circular optic edge. In some embodiments, the gusset region may have a larger radius than the optic edge, effectively providing an enlarged hinge, which can decrease distortion while maintaining or improving axial stability. Some embodiments of the gusset region may have a swept section. For example, the geometry may be swept about a center of rotation that is opposite the optical axis of the lens, which can provide a junction between the gusset region and the optic that has an arc that is flatter than the optic edge. The gusset may have a slanted portion that can blend into the anterior optic surface, and the placement of this transition may vary based on the power and optical profile of the optical surface. Additionally, or alternatively, some embodiments of the gusset region may have a cross-sectional shape that can improve axial stability without increasing the volume of the lens.

More generally, some embodiments comprise a lens for implanting into an eye, which may comprise an optic having an optical axis, an optic edge defined by a first arc having a first center that is coincident with the optical axis, and a haptic junction defined by a second arc having second center. The optical axis may be located between the second center and the second arc. A gusset may be coupled to the haptic junction, and a haptic may be coupled to the gusset. The gusset may have a thickness that increases between the haptic junction and the haptic. The optic edge may intersect the haptic junction. Additionally, or alternatively, the thickness of the gusset may increase monotonically with distance from the optical axis. In some embodiments, the thickness of the gusset may increase linearly with distance from the optical axis.

In some aspects, the first arc may have a first curvature, the second arc may have a second curvature, and the second curvature may be less than the first curvature. In yet other aspects, the first arc may have a first radius of curvature, the second arc may have a second radius of curvature, and the second radius of curvature may be larger than the first radius of curvature.

In more particular embodiments, the optic may comprise an anterior optic surface and a posterior optic surface. The optic edge may join at least a portion of the anterior optic surface and the posterior optic surface, and the haptic junction may join at least a portion of the anterior optic surface and the posterior optic surface.

Other embodiments of a lens may comprise an optic having an optical axis, an optic edge having a first center of curvature and a first radius of curvature, and a haptic junction having a second center of curvature and a second radius of curvature. The first center of curvature may be coincident with the optical axis and located between the second center of curvature and the haptic junction, and the second radius of curvature may be larger than the first radius of curvature. In more particular embodiments, the optic may comprise an anterior optic surface and a posterior optic surface. The optic edge may join at least a portion of the anterior optic surface and the posterior optic surface, and the haptic junction may join at least a portion of the anterior optic surface and at least a portion of the posterior optic surface.

In yet other embodiments, a lens may comprise an optic having an optical axis, an anterior optic surface, and a posterior optic surface; an optic edge having an anterior optic boundary that joins a portion of the anterior optic surface and a posterior optic boundary that joins a portion of the posterior optic surface; and a haptic junction having an anterior junction boundary that joins a portion of the anterior optic surface and a posterior junction boundary that joins a portion of the posterior optic surface. The anterior optic boundary may be defined by an anterior optic center of curvature and an anterior optic radius of curvature, and the posterior optic boundary may be defined by a posterior optic center of curvature and a posterior optic radius of curvature. The anterior junction boundary may be defined by an anterior junction center of curvature and an anterior junction radius of curvature, and the posterior junction boundary may be defined by a posterior junction center of curvature and a posterior junction radius of curvature. The anterior optic center of curvature and the posterior optic center of curvature may be coincident with the optical axis. The optical axis may be located between the anterior junction center of curvature and the posterior junction center of curvature. The anterior junction radius of curvature may be larger than the anterior optic radius of curvature, and the posterior junction radius of curvature may be larger than the posterior optic radius of curvature.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is an isometric view of an example of a lens, illustrating various features that may be associated with some embodiments.

FIG. 2 is a top view of the lens of FIG. 1.

FIG. 3 is a front view of the lens of FIG. 1.

FIG. 4 is a section view of the lens of FIG. 2.

FIGS. 5A-5B are schematic diagrams illustrating an example procedure of implanting the lens of FIG. 1 into an eye.

FIG. 6 is a diagram illustrating the axial displacement of an optic along an optical axis of a representative model of the lens of FIG. 1.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting

The example embodiments may also be described herein with reference to spatial relationships between various elements or to the spatial orientation of various elements depicted in the attached drawings. In general, such relationships or orientation assume a frame of reference consistent with or relative to a patient in a position to receive an implant. However, as should be recognized by those skilled in the art, this frame of reference is merely a descriptive expedient rather than a strict prescription.

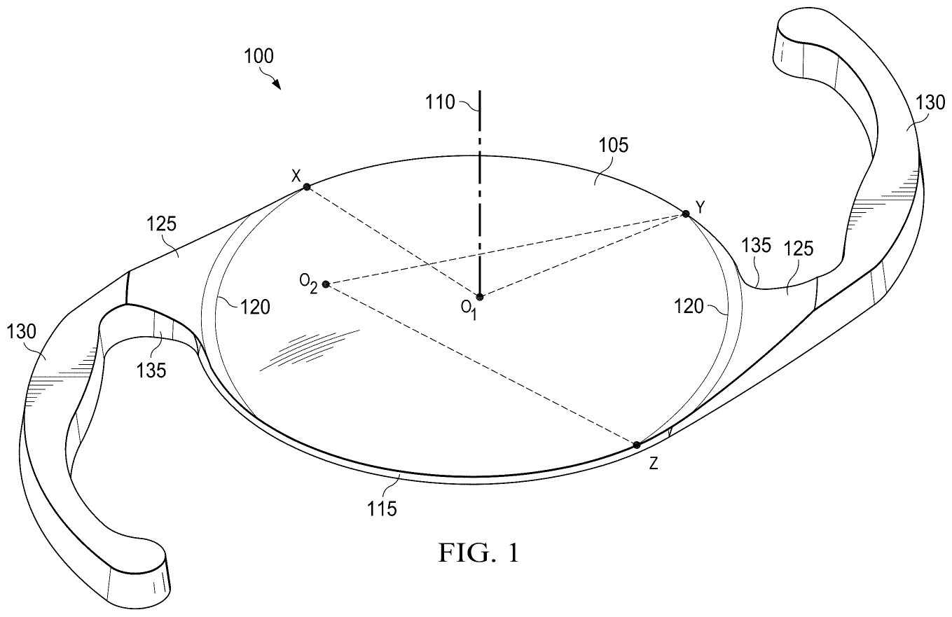

FIG. 1 is an isometric view of an example of a lens 100, illustrating various features that may be associated with some embodiments. As illustrated in the example of FIG. 1, the lens 100 may include an optic 105 having an optical axis 110, an optic edge 115, and a haptic junction 120; a gusset 125 coupled to the haptic junction 120; and a haptic 130 coupled to the gusset 125.

The haptic 130 may be an open-loop haptic, which generally comprises a proximal end coupled to the gusset 125 and a free distal end, as illustrated in the example of FIG. 1. The haptic 130 may also be curved, which is sometimes referred to as a “C-loop” or having a “C” shape. In some embodiments, the gusset 125 may have an elbow region 135, which can increase the flexibility of the haptic 130, particularly in the plane of the optic 105. The number of haptic junctions and haptics may vary. For example, as shown in FIG. 1, some embodiments of the lens 100 may have more than one haptic junction 120 and more than one haptic 130. In general, there is one haptic junction for each haptic, which may be symmetrical about the optical axis 110.

In some examples, the optic 105 and the haptic 130 may be molded in a single piece from the same material. The material used to make the lens 100 may be any soft, biocompatible material capable of being folded. Suitable materials may include hydrogel, silicone, or acrylic materials.

Geometrically, the optic axis 110 is understood to be an imaginary axis that passes through the center of the optic 105, and the optic edge 115 is a surface that may form an external perimeter of the optic 105. For example, the optic edge 115 of FIG. 1 forms an external perimeter of the optic 105, exclusive of the haptic junctions 120. In some examples, the optic edge may be defined as a curvilinear surface, such as a conical or cylindrical surface. Some examples of the optic edge 115 may comprise a surface that is curved in more than one dimension. For example, the optic edge 115 may have a toric profile in some embodiments. Similarly, the haptic junction 120 may form an internal boundary between the optic 105 and the haptic 130, exclusive of the optic edge 115. The haptic junction 120 may form an external perimeter of the optic 105 if not coupled to a haptic 130, so that the optic edge 115 and the haptic junction 120 provide a continuous perimeter.

The optic edge 115 may be generally defined, at least in part, by a first curve having a first center of curvature, and the haptic junction 120 may be defined, at least in part, by a second curve having second center of curvature. In FIG. 1, for example, the first curve is an arc between a point X and a point Y, having a first center of curvature O₁, and the haptic junction 120 is an arc between the point Y and the point Z, having a second center of curvature O₂. In some embodiments, a suitable angle of the arc XY may be in a range of about 80-90 degrees, and a suitable angle of the arc YZ may be in a range of about 60-70 degrees. The optic edge 115 and the haptic junction 120 may intersect at several points, such as at point Y and point Z of FIG. 1. More generally, each haptic junction may intersect the optic edge 115 at two points. The first center of curvature O₁ may be coincident with the optical axis 110, as illustrated in FIG. 1.

Additionally, or alternatively, some examples of the gusset 125 may have a thickness that increases between the haptic junction 120 and the haptic 130, as illustrated in FIG. 1.

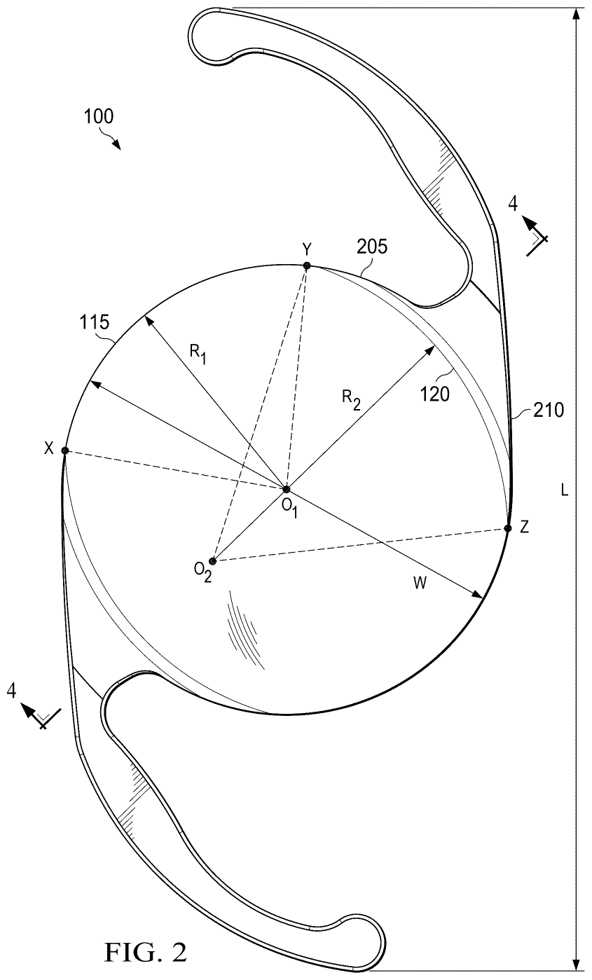

FIG. 2 is a top view of the lens 100 of FIG. 1, illustrating additional details that may be associated with some embodiments. For example, as illustrated in FIG. 2, the optic edge 115 (as defined by the first curve XY) may have a first radius of curvature R₁, and the haptic junction 120 (as defined by the second curve YZ) may have a second radius of curvature R₂. In the lens 100 of FIG. 2, the second radius of curvature R₂ is larger than the first radius of curvature R₁, and the first center of curvature O₁ (and thus the optical axis 110) is located between the second center of curvature O₂ and the second curve YZ. Consequently, the curvature of the haptic junction 120 is less than the curvature of the optic edge 115 of FIG. 2.

In some examples, the gusset 125 may have a portion that joins the haptic junction 120 along the full length of the second curve YZ, as illustrated in FIG. 2. As illustrated in FIG. 2, some examples of the gusset 125 may also have an edge 205 that intersects with the haptic junction 120 at point Y and an edge 210 that intersects with the haptic junction 120 at point Z. In some examples, the edge 205 may be curved, and in more particular examples, at least a portion of the edge 205 may comprise a curve defined by the center of curvature O₁ and the radius of curvature R₁ and may be adjacent to the optic edge 115. Additionally, or alternatively, the edge 210 may be tangent to the optic edge 115. For example, the edge 210 of FIG. 2 is tangent to the optic edge at the point where the optic edge 115 and the haptic junction 120 intersect (e.g., at point Z).

As illustrated in FIG. 2, the lens 100 generally has a length L, which may be measured between the extremities of the lens 100. In the example of FIG. 2, the length L is measured between the distal ends of the two haptics. The length L may vary, but a length L in a range of about 10 mm to about 15 mm may be suitable for many applications, and a length L of about 13.5 mm may be advantageous for some embodiments.

The lens 100 may have a width W that can vary as appropriate to achieve a desired outcome for particular applications. A width W of about 5 millimeters to about 8 millimeters may be advantageous for some embodiments. In more particular examples, a width W of about 6.5millimeters to about 7.5 millimeters may be suitable. In some examples, the width W may be twice the radius of curvature R₁ (i.e., W=2R₁).

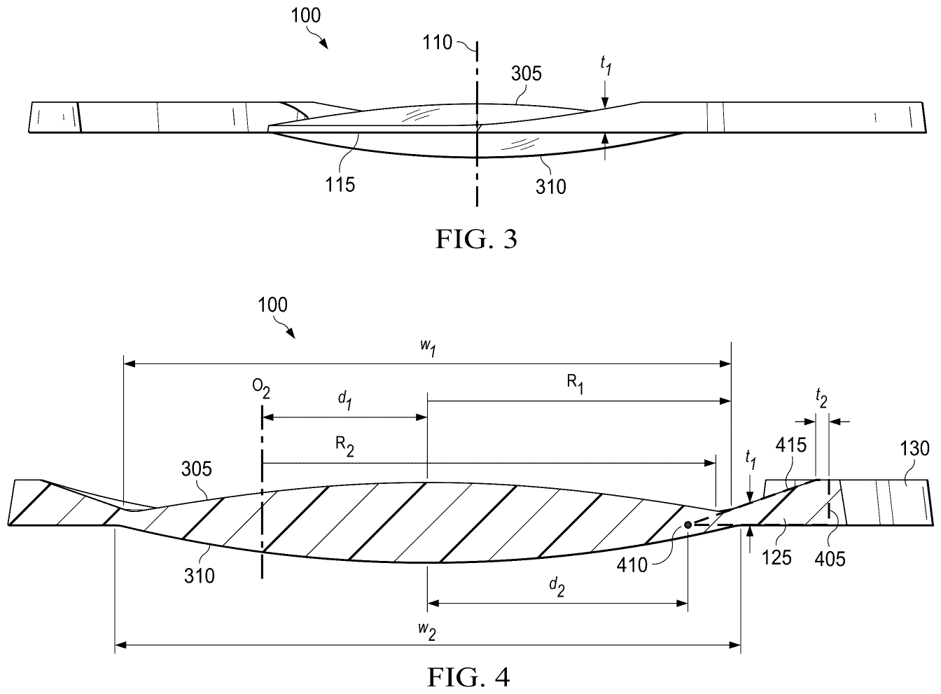

FIG. 3 is a front view of the lens 100 of FIG. 1, illustrating additional details that may be associated with some embodiments. For example, the lens 100 may have an anterior optic surface 305 and a posterior optic surface 310. The anterior optic surface 305, the posterior optic surface 310, or both may have any suitable profiles configured to correct a patient's vision. For example, either or both may be spheric, aspheric, toric, refractive, diffractive, or any suitable combination thereof. Additionally, or alternatively, either or both may extend to the optic edge 115 or may extend to a transition region (not shown in FIG. 3) between the optic edge 115 and the anterior optic surface 305 and/or the posterior optic surface 310.

As illustrated in the example of FIG. 3, the optic edge 115 may form an external perimeter around at least a portion of the optic 105 and join at least a portion of the anterior optic surface 305 and the posterior optic surface 310. More generally, the optic edge 115 may be a surface that extends between the anterior optic surface 305 and the posterior optic surface 310. In some examples, the optic edge 115 may comprise a continuously curved surface that joins the anterior optic surface 305 and the posterior optic surface 310. In such examples, the continuously curved surface may not include any tangents parallel to the optical axis 110, which may advantageously reduce the incidence of positive dysphotopsia results, at least in part, from edge glare.

In certain embodiments, the gusset 125 may have a thickness t₁ that monotonically increases with distance from the optical axis 110. For example, the thickness t₁ may be a minimum at the point of connection between the gusset 125 and the haptic junction 120, and the thickness t₁ may be a maximum at the point of connection between the gusset 125 and the haptic 130. In other examples, the thickness t₁ may increase over only a portion of the gusset 125. In more specific examples, the thickness t₁ may monotonically increase over a first distance from the optical axis 110, and the thickness t₁ may be constant or decrease (or a combination thereof) over a second distance from the optical axis 110.

FIG. 4 is a section view of the lens 100 taken along line 4-4 of FIG. 2, illustrating additional details that may be associated with some embodiments. For example, the anterior optic surface 305 may have a width w₁, and the posterior optic surface 310 may have a width w₂. In some examples, a range of about 4.5 millimeters to about 7.0 millimeters for either or both the width w₁ and the width w₂ may be advantageous. In some embodiments, the width w₁ and the width w₂ may not be equal. For example, the width w₁ may be less than the width w₂, as shown in the lens 100 of FIG. 4. In more particular examples, the width w₁ may be about 6 millimeters and the width w₂ may be about 6.15 millimeters.

FIG. 4 also illustrates additional geometric details that may be associated with some embodiments. For example, FIG. 4 further illustrates an example location of the second center of curvature O₂. In the example of FIG. 4, the second center of curvature O₂ is not coincident with the optical axis 110; it is located a distance d₁ from the optical axis 110 opposite the haptic junction 120 having the center of curvature O₂.

In general, the width W (see FIG. 2) is equivalent to the larger of the width w₁ and the width w₂, and the width w₁ is twice the radius of curvature R₁. If the width w₁ and the width W₂ are equal, then the width W is equal to both w₁ and w₂ (i.e., W=w₁=w₂). If the width w₁ and the width w₂ are not equal, then the boundary between the anterior optic surface 305 and the optic edge 115 may be defined by the first curve XY, having the first center of curvature O₁ and the first radius of curvature R₁ (as shown in FIG. 2), and the boundary between the posterior optic surface 310 and the optic edge 115 may be defined by a concentric curve having the first center of curvature O₁. For example, the radius of curvature of the concentric curve can be extended to intersect with the posterior optic surface 310 (e.g., equal to R₁+0.5(w₂−w₁)). Similarly, the boundary between the anterior optic surface 305 and the haptic junction 120 may be defined by the second curve YZ, having the second center of curvature O₂ and the second radius of curvature R₂ (as shown in FIG. 2), and the boundary between the posterior optic surface 310 and the haptic junction 120 may be defined by a concentric curve having the second center of curvature O₂. For example, the radius of curvature of the concentric curve can be extended to intersect with the posterior optic surface 310 (e.g., equal to R₂₊0.5(w₂−w₁)).

In some embodiments, the cross-section of the gusset 125 can be modeled as a right triangle revolved about the optical axis 110. An example of this triangular cross-section of the gusset 125 is illustrated in the example of FIG. 4, wherein the triangle has a first edge 405 located at the junction between the gusset 125 and the haptic 130, and a first vertex 410 of the triangle is located a distance d₂ from the optical axis 110. A distance d₂ of about 2.4 millimeters to about 2.5 millimeters may be suitable for some embodiments, and a distance d₂ of about 2.48 millimeters may be advantageous. The example triangle of FIG. 4 also has a hypotenuse 415 that intersects with the haptic junction 120 (as defined by the radius of curvature R₂). The location of the intersection between the hypotenuse 415 and the haptic junction 120 can vary based on the power and optical profile of the anterior optic surface 305. As shown in the example of FIG. 4, some embodiments of the gusset 125 may have a thickness t₁ that can increase linearly with distance from the haptic junction 120. In the example of FIG. 4, the gusset 125 also has a transition region between the first edge 405 and the haptic 130, the transition region having a thickness t₂.

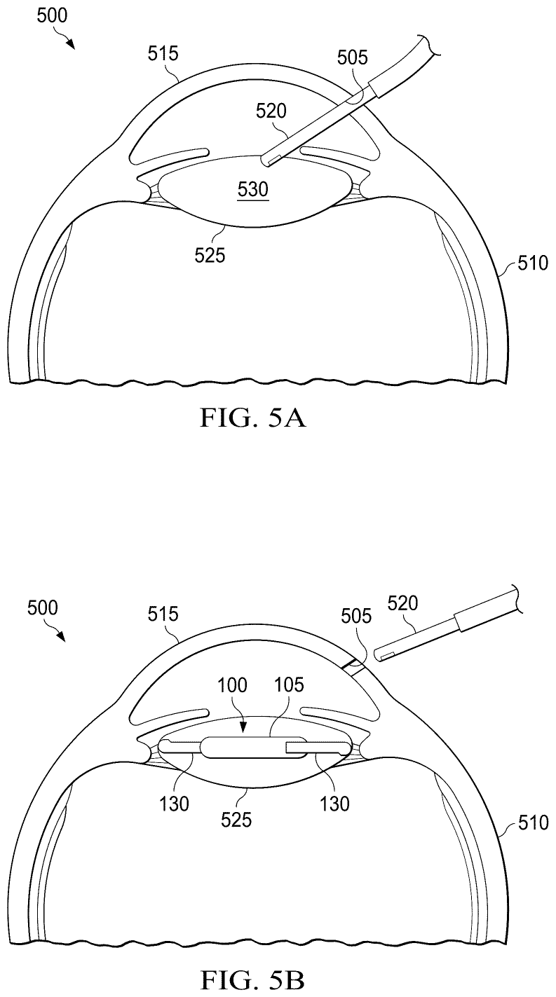

FIGS. 5A-5B are schematic diagrams illustrating an example procedure of implanting the lens 100 into an eye 500. As illustrated, an incision 505 may be made in the eye 500, for example. In some instances, the incision 505 may be made through the sclera 510 of the eye 500. In other instances, an incision may be formed in the cornea 515 of the eye 500. The incision 505 may be sized to permit insertion of a portion of a nozzle 520 to deliver the lens 100 into the capsular bag 525. For example, in some instances, the size of the incision 505 may have a length less than about 3000 microns (3 millimeters). In other instances, the incision 505 may have a length of from about 1000 microns to about 1500 microns, from about 1500 microns to about 2000 microns, from about 2000 microns to about 2500 microns, or from about 2500 microns to about 3000 microns.

After the incision 505 is made, the nozzle 520 can be inserted through the incision 505 so that the tip of the nozzle 520 aligns with the incision 505, allowing the nozzle 520 to extend into an interior portion 530 of the eye 500. The lens 100 can then be ejected through the nozzle 520 into the capsular bag 525 of the eye 500.

In some applications, the lens 100 may be delivered in a folded, straightened, or splayed configuration and can revert to an initial, resting state, within the capsular bag 525, as shown in FIG. 5B. The capsular bag 525 can retain the lens 100 within the eye 500 in a relationship relative to the eye 500 so that the optic 105 refracts light directed to the retina (not shown). The haptics 130 can engage the capsular bag 525 to secure the lens 100 therein. After dispensing the lens 100 into the capsular bag 525, the nozzle 520 may be removed from the eye 500 through the incision 505, and the eye 500 can be allowed to heal over time.

Haptic compression generally occurs during delivery and fixation of the lens 100 into an eye as the haptics conform to the capsular bag, such as in the example illustrated with respect to FIGS. 5A-5B. In a free state, the axial displacement is generally zero, particularly if the lens 100 is a single-body object since the haptics and the optic are effectively co-planar, but haptic compression can cause displacement of the optic along the optic axis. The resulting optic position at 10 millimeters can be significant since the capsular bag generally has a diameter between 10 millimeters and 11 millimeters. The resulting power of the optic can be affected if the optic is displaced excessively during haptic compression, which can lead to negative visual outcomes. The haptic junction 120 can provide a hinge to maintain stability, significantly reducing or eliminating such negative effects, while allowing the thickness of the lens (i.e., the distance between the anterior optic surface 305 and the posterior optic surface 310) to be reduced.

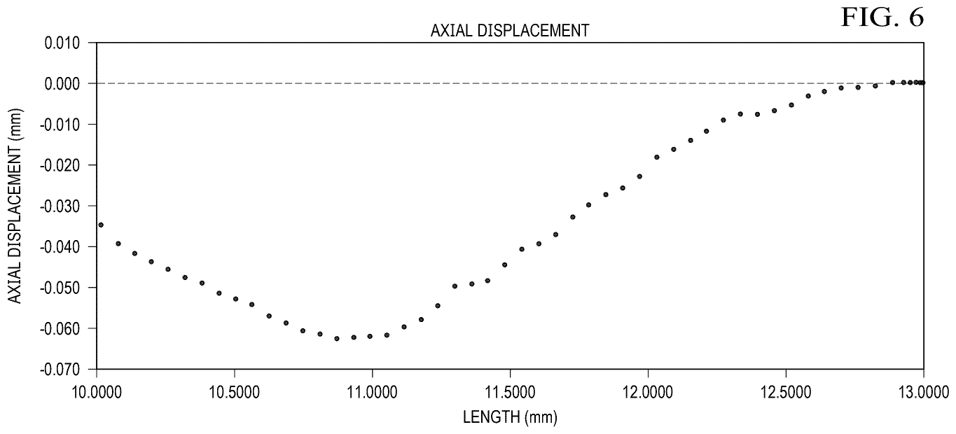

For example, FIG. 6 is a plot of data based on an analysis of a finite element model representative of an embodiment of the lens 100 of FIG. 1, illustrating the axial displacement of the optic 105 along the optical axis 110 as a function of haptic compression (i.e., the decrease in the length L). More particularly, FIG. 6 demonstrates the results of using an ANSYS finite element analysis model of the lens 100, wherein the thickness of the lens 100 is reduced so that the lens 100 has a volume of about 14.78 cubic millimeters and compression is simulated from a length L of about 13 millimeters (a free state) down to a length L of about 10 millimeters. FIG. 6 demonstrates good results of axial displacement of the optic along the optical axis, being about 35 microns at the compressed length L of about 10 millimeters. The results are significant and surprising since the various features of the lens 100 described herein can allow the thickness (and thus the volume) of the lens 100 to be reduced while maintaining axial stability comparable to a lens having a larger thickness, which in turn can allow smaller incisions for delivery while maintaining positive visual outcomes.

While shown in a few illustrative embodiments, a person having ordinary skill in the art will recognize that the systems, apparatuses, and methods described herein are susceptible to various changes and modifications that fall within the scope of the appended claims.

Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A lens for implanting into an eye, the lens comprising:
an optic having an optical axis;
an optic edge defined by a first arc having a first center that is coincident with the optical axis;
a haptic junction defined by a second arc having second center, the optical axis being between the second center and the second arc;
a gusset coupled to the haptic junction; and
a haptic coupled to the gusset;
wherein the gusset has a thickness that increases between the haptic junction and the haptic.

2. The lens of claim 1, wherein the optic edge intersects the haptic junction.

3. The lens of claim 1, wherein:
the first arc has a first curvature;
the second arc has a second curvature; and
the second curvature is less than the first curvature.

4. The lens of claim 1, wherein:
the first arc has a first radius of curvature;
the second arc has a second radius of curvature; and
the second radius of curvature is larger than the first radius of curvature.

5. The lens of claim 1, wherein the thickness of the gusset increases monotonically with distance from the optical axis.

6. The lens of claim 1, wherein the thickness of the gusset increases linearly.

7. The lens of claim 1, wherein:
the optic comprises an anterior optic surface and a posterior optic surface;
the optic edge joins at least a portion of the anterior optic surface and the posterior optic surface; and
the haptic junction joins at least a portion of the anterior optic surface and the posterior optic surface.

8. The lens of claim 7, wherein:
the anterior optic surface has a first width;
the posterior optic surface has a second width;
the first width is less than the second width;
the first arc defines a boundary that joins the anterior optic surface; and
the second arc defines a boundary that joins the anterior optic surface.

9. The lens of claim 1, wherein the haptic is an open-loop haptic.

10. A lens for implanting into an eye, the lens comprising:
an optic having an optical axis;
an optic edge having a first center of curvature and a first radius of curvature; and
a haptic junction having a second center of curvature and a second radius of curvature;
wherein the first center of curvature is coincident with the optical axis and located between the second center of curvature and the haptic junction, and the second radius of curvature is larger than the first radius of curvature.

11. The lens of claim 10, wherein:
the optic comprises an anterior optic surface and a posterior optic surface;
the optic edge joins at least a portion of the anterior optic surface and the posterior optic surface; and
the haptic junction joins at least a portion of the anterior optic surface and joins at least a portion of the posterior optic surface.

12. The lens of claim 10, wherein:
the optic comprises an anterior optic surface and a posterior optic surface;
the optic edge joins at least a portion of the anterior optic surface and the posterior optic surface;
the haptic junction joins at least a portion of the anterior optic surface and joins at least a portion of the posterior optic surface; and
the anterior optic surface has a width that is twice the first radius of curvature.

13. The lens of claim 10, wherein:
the optic comprises an anterior optic surface and a posterior optic surface;
the optic edge joins at least a portion of the anterior optic surface and the posterior optic surface;
the haptic junction joins at least a portion of the anterior optic surface and joins at least a portion of the posterior optic surface;
the anterior optic surface has a first width;
the posterior optic surface has a second width; and
the first width is equal to the second width.

14. The lens of claim 10, wherein:
the optic comprises an anterior optic surface and a posterior optic surface;
the optic edge joins at least a portion of the anterior optic surface and the posterior optic surface;
the haptic junction joins at least a portion of the anterior optic surface and joins at least a portion of the posterior optic surface;
the anterior optic surface has a first width that is twice the first radius of curvature;
the posterior optic surface has a second width; and
the first width is equal to the second width.

15. The lens of claim 10, wherein:
the optic comprises an anterior optic surface and a posterior optic surface;
the optic edge joins at least a portion of the anterior optic surface and the posterior optic surface;
the haptic junction joins at least a portion of the anterior optic surface and joins at least a portion of the posterior optic surface;
the anterior optic surface has a first width;
the posterior optic surface has a second width; and
the first width is less than the second width.

16. The lens of claim 10, wherein:
the optic comprises an anterior optic surface and a posterior optic surface;
the optic edge comprises a first optic boundary that joins at least a portion of the anterior optic surface and a second optic boundary that joins at least a portion of the posterior optic surface;
the haptic junction comprises a first junction boundary that joins at least a portion of the anterior optic surface and a second junction boundary that joins at least a portion of the posterior optic surface;
the anterior optic surface has a first width;
the posterior optic surface has a second width;
the first width is less than the second width;
the first optic boundary is defined by the first center of curvature and the first radius of curvature; and
the first junction boundary is defined by the second center of curvature and the second radius of curvature.

17. The lens of claim 10, wherein:
the optic comprises an anterior optic surface and a posterior optic surface;
the optic edge comprises a first optic boundary that joins at least a portion of the anterior optic surface and a second optic boundary that joins at least a portion of the posterior optic surface;
the haptic junction comprises a first junction boundary that joins at least a portion of the anterior optic surface and a second junction boundary that joins at least a portion of the posterior optic surface;
the anterior optic surface has a first width that is twice the first radius of curvature;
the posterior optic surface has a second width;
the first width is less than the second width;
the first optic boundary is defined by the first center of curvature and the first radius of curvature; and
the first junction boundary is defined by the second center of curvature and the second radius of curvature.

18. A lens for implanting into an eye, the lens comprising:
an optic having an optical axis, an anterior optic surface, and a posterior optic surface;
an optic edge having an anterior optic boundary that joins a portion of the anterior optic surface and a posterior optic boundary that joins a portion of the posterior optic surface; and
a haptic junction having an anterior junction boundary that joins a portion of the anterior optic surface and a posterior junction boundary that joins a portion of the posterior optic surface;
wherein the anterior optic boundary is defined by an anterior optic center of curvature and an anterior optic radius of curvature;
wherein the posterior optic boundary is defined by a posterior optic center of curvature and a posterior optic radius of curvature;
wherein the anterior junction boundary is defined by an anterior junction center of curvature and an anterior junction radius of curvature;
wherein the posterior junction boundary is defined by a posterior junction center of curvature and a posterior junction radius of curvature;
wherein the anterior optic center of curvature and the posterior optic center of curvature are coincident with the optical axis;
wherein the optical axis is located between the anterior junction center of curvature and the posterior junction center of curvature;
wherein the anterior junction radius of curvature is larger than the anterior optic radius of curvature; and
wherein the posterior junction radius of curvature is larger than the posterior optic radius of curvature.

19. The lens of claim 18, wherein the anterior junction boundary intersects the anterior optic boundary, and the posterior junction boundary intersects the posterior optic boundary.

20. The lens of claim 18, further comprising:
a gusset coupled to the haptic junction; and
a haptic coupled to the gusset;
wherein the gusset has a thickness that increases between the haptic junction and the haptic.

21. (canceled)
